(19) United States
(12) Patent Application Publication
(10) Pub. No.: US 2025/0243142 A1
(43) Pub. Date: Jul. 31, 2025

(54) PROCESS FOR PRODUCTION OF PROPYLENE OXIDE AND STYRENE MONOMER

(71) Applicant: Lyondell Chemical Technology, L.P., Houston, TX (US)

(72) Inventors: Karl P. Rufener, Humble, TX (US); Sunti Kongkitisupchai, Houston, TX (US); Kimberly A. Petry, Friendswood, TX (US); Anthony S. Dearth, Kingwood, TX (US)

(73) Assignee: Lyondell Chemical Technology, L.P., Houston, TX (US)

(21) Appl. No.: 19/040,297

(22) Filed: Jan. 29, 2025

Related U.S. Application Data
(60) Provisional application No. 63/626,635, filed on Jan. 30, 2024.

(51) Int. Cl. C07C 29/84; B01D 3/14; C07C 5/05; C07C 45/83

(52) U.S. Cl. CPC C07C 29/84; B01D 3/141; B01D 3/143; C07C 5/05; C07C 45/83

(57) ABSTRACT

Disclosed is the use of a dividing-wall column in a process for co-producing propylene oxide and styrene monomer. Feed streams to a dividing-wall column comprise a bottoms product produced in recovering crude propylene oxide from an epoxidation reaction product, a hydrogenated byproduct styrene monomer production, and a light fraction of a bottoms product withdrawn from the dividing-wall column. Products from the dividing-wall column comprise an overhead product comprising ethylbenzene for recycle to an oxidation zone, an intermediate product comprising methylbenzyl alcohol and acetophenone as feed to a dehydration zone to produce styrene monomer, and a bottoms product comprising methylbenzyl alcohol and heavy materials.

### CROSS-REFERENCE TO RELATED APPLICATIONS

The application claims the benefit of priority to U.S. Provisional Patent Application No. 63/626,635 filed on Jan. 30, 2024, which is incorporated herein by reference in its entirety.

### FIELD OF THE INVENTION

This disclosure generally relates to processes and systems for the co-production of propylene oxide and styrene monomer.

### BACKGROUND OF THE INVENTION

Methods of co-producing propylene oxide and styrene monomer (“POSM” processes) include the oxidation of ethyl benzene to form ethyl benzene hydroperoxide, the catalytic reaction of the hydroperoxide with propylene to form propylene oxide and 1-phenyl ethanol, and the dehydration of the 1-phenyl ethanol (also known as α-methylbenzyl alcohol) to produce styrene monomer. An example of a POSM process is described at U.S. Pat. No. 3,351,635. POSM processes also are described at U.S. Pat. Nos. 5,210,354 and 5,276,235, and U.S. Pat. App. Pub. No. 2018/0221787. Each of these references are incorporated herein by reference.

Many advances have been made in the POSM processing. However, there is still a need for further development of processes to increase capacity and/or efficiency of such processes. Ideally, such improved processes could be implemented in existing by retrofitting or built into new facilities employing commonly used equipment and familiar techniques.

### SUMMARY OF THE INVENTION

The present disclosure provides processes and systems for co-production of propylene oxide and styrene monomer. The processes and systems provide for use of a dividing-wall column to replace a cascade of two conventional distillation columns currently used to process recycle streams within a process for co-production of propylene oxide and styrene monomer. The dividing-wall column provides equivalent or substantially equivalent performance to the conventional two-column system such that the processes and systems disclosed herein are suitable for use in design of new facilities or for retrofitting existing facilities. The processes and systems using a dividing-wall column as disclosed herein provide reduced capital cost and/or operating cost as compared to a conventional two-column system.

Disclosed herein is a process comprising a dividing-wall column (DWC) having an upper section, an intermediate section, and a lower section. The intermediate section comprises a vertical partition which divides the intermediate section into a feed side and a product side. A first feed stream and a second feed stream are introduced to the feed side of the intermediate section of the DWC, wherein the first and second feed streams comprise ethylbenzene, methylbenzyl alcohol, and acetophenone. A third feed stream is introduced to the lower section of the DWC, wherein the third stream comprises methylbenzyl alcohol and acetophenone. Distillation conditions are implemented in the DWC to facilitate withdrawal of an overhead product stream, an intermediate product stream, and a bottoms product stream. The overhead product stream comprises ethylbenzene. The intermediate product stream comprises methylbenzyl alcohol and acetophenone and is withdrawn from the product side of the intermediate section. The bottoms stream comprises methylbenzyl alcohol and heavy materials.

In another aspect, disclosed herein is a system for producing propylene oxide and styrene monomer. In some embodiments, the system comprises an oxidation reactor, an oxidate concentration unit, a first distillation unit, and a dividing-wall distillation column. The oxidation reactor receives ethylbenzene and oxygen to produce a crude oxidate. The oxidate concentration unit receives the crude oxidate and discharges a concentrated oxidate. The epoxidation reactor receives the concentrated oxidate and propylene and discharges a propylene oxide-containing stream. The first distillation receives the propylene oxide-containing stream and discharges a light overhead stream comprising propylene oxide and unreacted propylene and a heavy bottoms stream comprising ethylbenzene, methylbenzyl alcohol, acetophenone, and heavy materials. The dividing-wall distillation column receives the heavy bottoms stream and discharges an overhead product stream, an intermediate product stream, and a bottoms product stream, wherein the overhead product stream comprises ethylbenzene, the intermediate product stream comprises methylbenzyl alcohol and acetophenone, and the bottoms product stream comprises methylbenzyl alcohol and heavy materials.

The process for producing propylene comprises obtaining a catalyst composition comprising a skeletal isomerization catalyst and a metathesis catalyst, wherein the weight ratio of the skeletal isomerization catalyst to the metathesis catalyst is from 1:2 to 10:1. The process further comprises adding ethylene and a feed stream comprising isobutylene to a reaction zone comprising the catalyst composition at a temperature in the range of from 400° C. to 550° C., under conditions sufficient to produce an effluent stream comprising propylene.

In some embodiments, the process further comprises separation of a C₂-stream and/or a C₄-C₅ hydrocarbon stream for recycling as feed to the reactor. In some embodiments, the process further comprises separation of a propylene stream and/or a C₆+ hydrocarbon stream as product streams from the process.

In some embodiments, a system for producing propylene comprises a reactor for isomerization and metathesis, the reactor having one or more inlets for receiving ethylene and a feed stream comprising isobutylene and one or more outlets for withdrawal of an effluent comprising C₂-C₆₊ hydrocarbons. The system further comprises a first distillation column having one or more inlets for receiving the effluent from the reactor, one or more overhead outlets for withdrawal of a first overhead stream comprising C₂-C₃ hydrocarbons, and one or more bottoms outlets for withdrawal of a first bottoms stream comprising C₄-C₆₊ hydrocarbons. The system further comprises a second distillation column having one or more inlets for receiving the first overhead stream from the first distillation column, one or more overhead outlets for withdrawal of a second overhead stream comprising one or more C₂ hydrocarbons, and one or more bottoms outlets for withdrawal of a second bottoms stream comprising one or more C₃ hydrocarbons. A fluid connection is provided from at least one of the one or more overhead outlets from the second distillation column to at least one of the one or more inlets to the reactor.

In some embodiments, the system further comprises a third distillation column having one or more inlets for receiving the first bottoms stream from the first distillation column, one or more outlets for withdrawal of a third overhead stream comprising C₄-C₅ hydrocarbons, and one or more outlets for withdrawal of a first bottoms stream comprising one or more C₆₊ hydrocarbons, including up to C₁₆ hydrocarbons. A fluid connection is provided from at least one of the one or more outlets from the third distillation column to at least one of the one or more inlets to the reactor.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other catalyst compositions and/or processes for carrying out the same purposes of the present invention. It should also be realized by those skilled in the art that such equivalent constructions do not depart from the spirit and scope of the invention as set forth in the appended claims. The novel features which are believed to be characteristic of the invention, both as to its compositions and methods, together with further objects and advantages will be better understood from the following description.

### BRIEF DESCRIPTION OF THE FIGURES

The claimed subject matter may be understood by reference to the following description taken in conjunction with the accompanying drawings, in which like reference numerals identify like elements, and in which:

FIG. 1 shows a simplified flow diagram of a process for co-producing propylene oxide and styrene monomer;

FIG. 2 shows a simplified flow diagram of the process the ethylbenzene/methylbenzyl alcohol recovery section 150 as shown in FIG. 1, wherein process comprises 2 distillation columns for recovery of ethylbenzene and methylbenzyl alcohol; and

FIG. 3 shows a simplified flow diagram of the process the ethylbenzene/methylbenzyl alcohol recovery section 150 as shown in FIG. 1, wherein process comprises a dividing-wall column for recovery of ethylbenzene and methylbenzyl alcohol, according to embodiments of the invention.

While the disclosed process and composition are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “a” or “an” when used in conjunction with the term “comprising” in the claims or the specification means one or more than one, unless the context dictates otherwise.

As used herein, “about” means the stated value plus or minus the margin of error of measurement or plus or minus 10% if no method of measurement is indicated.

As used herein, “comprise”, “have”, “include” and “contain” (and their variants) are open-ended linking verbs and allow the addition of other elements when used in a claim.

As used herein, “consisting essentially of” excludes additional material elements, but allows the inclusions of non-material elements that do not substantially change the nature of the invention.

As used herein, “consisting of” is closed and excludes all additional elements.

As used herein, “conversion” is used to denote the percentage of a component fed which disappears across a reactor.

As used herein, “distillation column” refers to a column that can separate a liquid mixture into its component parts or fractions by selective boiling and condensation. In a typical distillation, a liquid mixture is heated in the column wherein the resulting vapor rises up the column. The vapor condenses on trays inside the column, and returns to the bottom of the column, refluxing the rising distillate vapor. The more reflux and/or more trays provided, the better the column's separation of lower boiling materials from higher boiling materials. Sometimes, packing material is used in the columns (instead of trays) to improve contact between the two phases.

As used herein, “dividing-wall column” refers to a distillation column having a vertical partition baffle, or wall, between the feed zone and side draw to produce three high-purity products—overhead, bottoms, and intermediate. The partition baffle separates the feed from the intermediate product draw to minimize remixing inefficiency (which occurs when the feed stream contaminates the intermediate product) by fractionating the feed into higher-purity intermediate streams that are further separated on the other side of the partition. In some embodiments, there can be more than three product streams and more than one partition. Additional information regarding dividing-wall columns can be found in Vazzoler, Alex. (2022). An introduction to Dividing wall columns, design and modelling (DWC), Journal of Engineering Research, 2, 1, 10.22533/at.ed.317222230014 and Norbert Asprion, Gerd Kaibel, Dividing wall columns: Fundamentals and recent advances, Chemical Engineering and Processing: Process Intensification, Vol. 49, Iss. 2, 2010, Pages 139-146, ISSN 0255-2701, https://doi.org/10.1016/j.cep.2010.01.013, the substance of which are fully incorporated by reference herein.

As used herein, “heavies” or “heavy materials” are used to denote mixed hydrocarbons having a higher boiling point than methylbenzyl alcohol.

As used herein, “methylbenzyl alcohol” means α-methylbenzyl alcohol, also known as 1-phenyl ethanol.

As used herein, “or” in the claims is used to mean “and/or” unless explicitly indicated to refer to alternatives only or if the alternatives are mutually exclusive.

As used herein, “or” in the claims is used to mean “and/or” unless explicitly indicated to refer to alternatives only or if the alternatives are mutually exclusive.

As used herein, “overhead product” means the effluent exiting the top of a distillation tower. If the overhead product is in the form of a vapor it is referred to herein as a “vapor”; alternatively, overhead products in the form of a liquid are referred to herein as a “distillate” or “liquid distillate”. The distillate can be used for reflux back to the distillation tower.

All concentrations herein are by weight percent (“wt %”) unless otherwise specified.

### Co-Production of Propylene Oxide and Styrene Monomer

Disclosed herein is an improvement to a process for co-producing propylene oxide and styrene monomer (POSM process). In some embodiments, the POSM process comprises reacting ethylbenzene with oxygen and alkali in an oxidation reaction zone under conditions sufficient to form a product stream comprising hydroperoxide. In some embodiments, the reaction conditions comprise a temperature and pressure sufficient to produce a product stream comprising a desired concentration of hydroperoxide in the product stream without the use of a catalyst, as is known in the art. In some embodiments, reaction conditions in the oxidation zone comprise a pressure in the range of from 1 psia (34.5 kPa-a) to 1,000 psia (6.8 MPa-a) or from 30 psia (207 kPa-a) to 150 psia (1.03 MPa-a), a temperature in the range of from range 40° C. to 180° C. or from 90° C. to 150° C., or a combination thereof.

A portion of the product stream having an increased concentration of ethylbenzene hydroperoxide is recovered and introduced to an epoxidation reaction zone along with propylene under conditions sufficient to produce a reaction product comprising propylene oxide. In some embodiments, the reaction conditions comprise contacting the feed mixture with an epoxidation catalyst at a temperature and pressure sufficient to produce a desired concentration of propylene oxide, as is known in the art. In some embodiments, the epoxidation reaction carried out in the liquid phase in the presence of an effective dissolved catalytic amount of molybdenum, tungsten, titanium, columbium, tantalum, rhenium, selenium, chromium, zirconium, tellurium, or uranium catalyst. In some embodiments, reaction conditions in the epoxidation zone comprise a pressure in the range of from 1 psia (34.5 kPa-a) to 1,000 psia (6.8 MPa-a) or from 30 psia (207 kPa-a) to 700 psia (4.83 MPa-a), a temperature in the range of from range 20° C. to 120° C. or from 50° C. to 150° C., or a combination thereof.

The reaction product is distilled to remove unreacted propylene to produce crude propylene oxide product. The unreacted propylene can be conveniently recycled to the epoxidation reaction zone. The crude propylene oxide is then caustic washed and successively distilled to separately recover propylene oxide product, ethylbenzene (EB), a mixture of α-methylbenzyl alcohol (MBA) and acetophenone (ACP), and a heavy organic sodium-containing stream (heavies). The recovered EB can be conveniently recycled to the oxidation reaction zone.

Further details regarding processes for co-production of propylene oxide and styrene monomer are described in U.S. Pat. Nos. 3,351,635; 3,439,001; 4,066,706; 4,262,143; 5,210,354; 5,276,235; and 8,142,661; and U.S. Patent Application Publication Nos. 2019/0241534 and 2020/0354300; the disclosure of each of which is hereby incorporated herein in its entirety for all purposes not contrary to this disclosure.

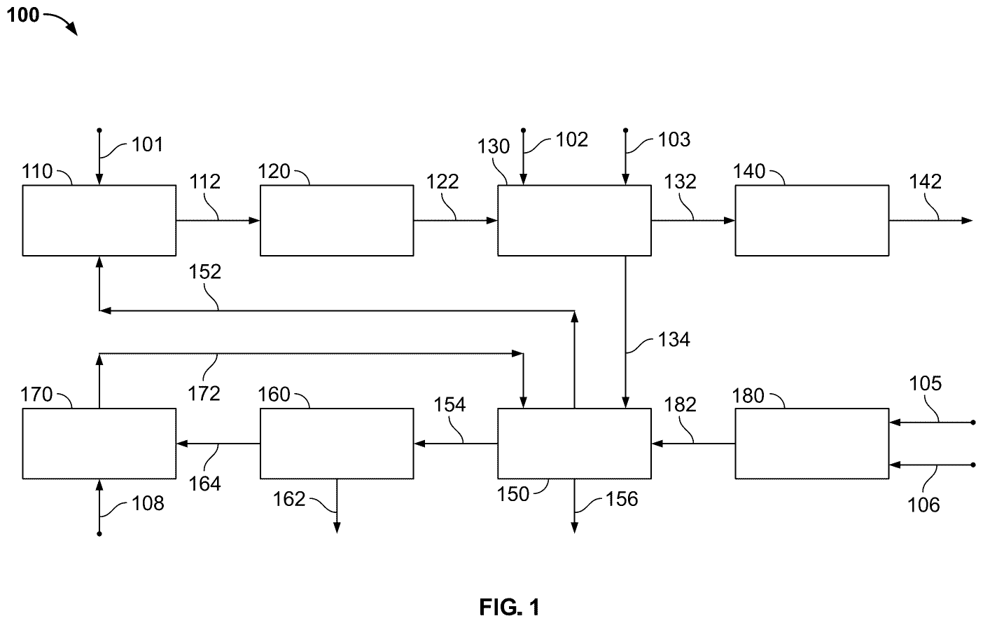

FIG. 1 shows a schematic representation of a process 100 for co-production of propylene oxide and styrene monomer. Ethylbenzene is fed to oxidation reactor 110 via line 152 and therein oxidized to ethylbenzene hydroperoxide by reaction with molecular oxygen which is introduced via line 101. The reaction mixture from oxidation reactor 110 comprised of unreacted ethylbenzene, ethylbenzene hydroperoxide, MBA, ACP and heavies passes via line 112 to oxidate concentration 120 comprised of distillation columns to produce a concentrated oxidate product and recover ethylbenzene for re-use in oxidation reactor 110. The concentrated oxidate product, comprising ethylbenzene hydroperoxide, passes to a propylene oxide production unit 130 comprising an epoxidation reactor and a distillation column. The ethylbenzene hydroperoxide and propylene, introduced via line 102, are contacted with catalyst, added via line 103, under reaction conditions in the reactor to form an epoxidation reaction product.

Further within the propylene oxide production unit 130, the epoxidation reaction product is treated with aqueous caustic to neutralize acidic materials and to remove the epoxidation catalyst to form a treated epoxidation product. The treated epoxidation product comprises unreacted propylene, propylene oxide, EB, MBA, ACP and heavy materials passes to a distillation column. An overhead mixture of unreacted propylene and product propylene oxide is withdrawn from the distillation column and is removed from the propylene oxide production unit 130 via line 132 and sent to purification processes 140, wherein the mixture is resolved into an unreacted propylene fraction for recycle and product propylene oxide which is further purified by conventional means and withdrawn via line 142.

A bottoms fraction comprised of ethyl benzene, MBA, ACP, and heavies is withdrawn from the distillation column and removed from the propylene oxide production unit 130 via line 134 (PO column bottoms) and passes to ethylbenzene/methylbenzyl alcohol (EB/MBA) recovery unit 150. An ethyl benzene stream is withdrawn from EB/MBA recovery unit 150 and is recycled to oxidation reactor 110 via line 152.

A second stream, comprising MBA and ACP is withdrawn from EB/MBA recovery unit 150 and is passed via line 154 and passed to dehydration zone 160, wherein MBA is converted to styrene monomer which is recovered via line 162. A byproduct stream comprising unreacted ACP is separated and removed from dehydration zone 160 and sent via line 164 to a hydrogenation zone 170, wherein hydrogen is added via line 108. The byproduct stream is hydrogenated to form a hydrogenate stream, wherein ACP is converted to MBA by known procedures. The hydrogenate stream comprising MBA is recycled to EB/MBA recovery unit 150 via line 172 (hydrogenate).

A third stream, comprising heavies, is withdrawn from EB/MBA recovery unit 150 and is sent via line 156 to heavy fuels blending or other disposition outside the process disclosed herein.

Ethylene and benzene are added to ethylbenzene unit 180 via lines 105 and 106, respectively. Ethylbenzene is withdrawn from ethylbenzene unit 180 and sent to EB/MBA recovery unit 150 via line 182.

### EB/MBA Recovery Unit

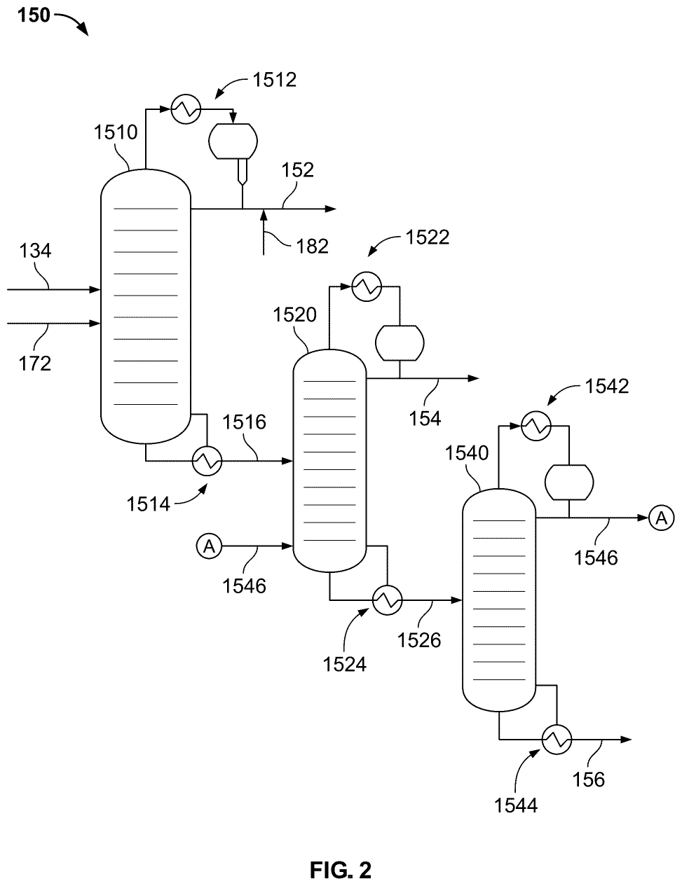

FIG. 2 provides a schematic representation of a portion of the process within EB/MBA recovery unit 150, shown in FIG. 1. Streams 134 and 172 are introduced into distillation column 1510. Distillation conditions in distillation column 1510 are controlled by heat removal from condenser loop 1512 and heat addition by reboiler loop 1514. The overhead stream withdrawn from distillation column 1510, comprising EB, is recycled via line 152 to the oxidation reactor 110, as shown in FIG. 1. Stream 182 from EB unit 180 also joins with stream 152. The bottoms stream from distillation column 1510, comprising MBA, ACP, and heavies passes via line 1516 to distillation column 1520.

Streams 1516 and 1546 (MBA stripper distillate) are introduced into distillation column 1520. Distillation conditions in distillation column 1520 are controlled by heat removal from condenser loop 1522 and heat addition by reboiler loop 1524. The overhead stream withdrawn from distillation column 1520, comprising MBA and ACP passes via line 154 to dehydration zone 160, as shown in FIG. 1. The bottoms stream from distillation column 1520, comprising MBA, ACP, and heavies passes via line 1526 to distillation column 1540.

Stream 1526 is introduced into distillation column 1540. Distillation conditions in distillation column 1540 are controlled by heat removal from condenser loop 1542 and heat addition by reboiler loop 1544. The overhead stream withdrawn from distillation column 1540, comprising MBA and ACP is recycled via line 1546 to distillation column 1520. The bottoms stream from distillation column 1540, comprising heavies, is sent via line 156 to heavy fuels blending or other disposition outside the process disclosed herein.

EB/MBA Recovery Unit with DWC

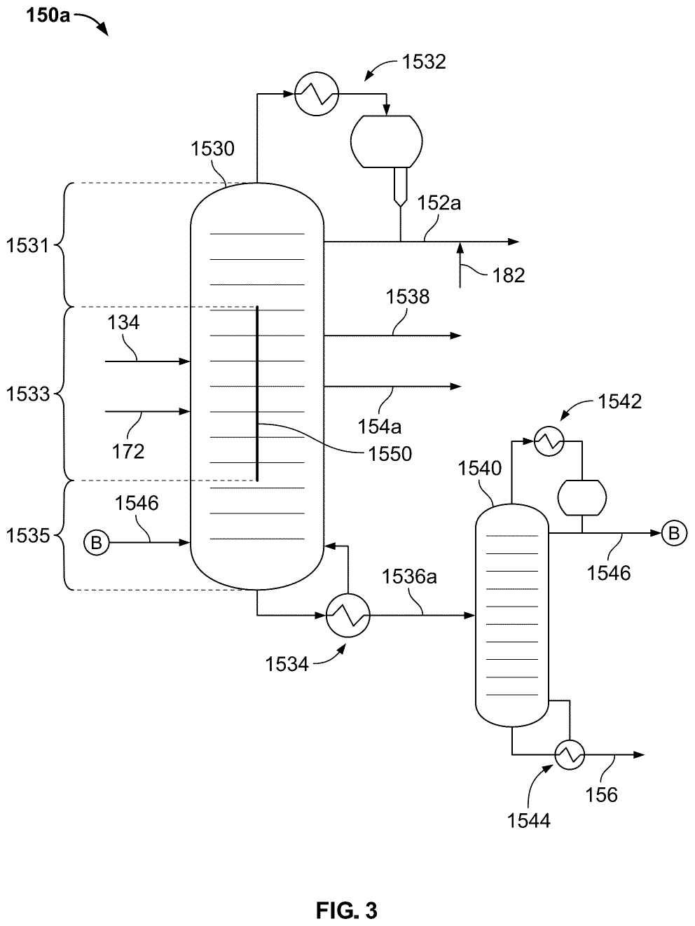

FIG. 3 provides a schematic representation of the process within EB/MBA recovery unit 150, wherein distillation columns 1510 and 1520, as shown in FIG. 2, are replaced with a divided wall column (DWC) 1530. Streams 134, 172, and 1546 are introduced into DWC 1530. Distillation conditions in distillation column 1530 are controlled by heat removal from condenser loop 1532 and heat addition by reboiler loop 1534. The overhead stream withdrawn from distillation column 1530, comprising EB, is recycled via line 152a to the oxidation reactor 110, as shown in FIG. 1. Stream 182 from EB unit 180 also joins with stream 152a. The side stream withdrawn from distillation column 1530, comprising MBA and ACP passes via line 154a to dehydration zone 160, as shown in FIG. 1. The bottoms stream from distillation column 1530, comprising MBA, ACP, and heavies passes via line 1536a to distillation column 1540.

Stream 1536a is introduced into distillation column 1540. Distillation conditions in distillation column 1540 are controlled by heat removal from condenser loop 1542 and heat addition by reboiler loop 1544. The overhead stream withdrawn from distillation column 1540, comprising MBA and ACP is recycled via line 1546 to distillation column 1530. The bottoms stream from distillation column 1540, comprising heavies, is sent via line 156 to heavy fuels blending or other disposition outside the process disclosed herein.

### Dividing-Wall Column Distillation

FIG. 3 provides a schematic representation of the process within EB/MBA recovery unit 150a, comprising a dividing-wall column (DWC). EB/MBA recovery unit 150a is intended to exemplify a direct replacement for the EB/MBA recovery unit 150 shown in FIG. 2, wherein DWC 1530 in FIG. 3 provides equivalent or substantially equivalent service duty to the combination of columns 1510 and 1520 in FIG. 2.

In some embodiments, a dividing-wall column (DWC) has an upper section 1531, an intermediate section 1533, and a lower section 1535 as shown in FIG. 3. The intermediate section 1533 comprises a vertical partition 1550, which divides the intermediate section into a feed side and a product side. The partition 1550 divides the intermediate section 1533 such that the feed side and the product side are fluidly connected only by fluid and/or vapor flow through the upper and/or lower sections of the DWC.

In some embodiments, DWC comprises trays, packing, or combination thereof sufficient to implement theoretical stages in the range of from 12 to 60, from 22 to 45, from 24 to 40, or from 26 to 35. One of ordinary skill in the art would recognize how to select specific ranges of theoretical stages to satisfy typical product production and quality requirements. Selection of ranges depends on feed quality and desired purity of product streams. Overall capacity is primarily determined by hydraulic limitations, such as, but not limited to column diameter and tray flow limits. The partition 1550 extends to span across 10 to 20, across 11 to 18, or across 12 to 16 of these theoretical stages. One of ordinary skill in the art would recognize how to select the position of the partition, more specifically the upper end and the lower end of the partition, to adjust separation performance for typical variations in feed quality or quantity.

In some embodiments, the upper section comprises from 25% to 50% of the theoretical trays of the column, the intermediate section comprises from 30% to 70% of the theoretical trays of the column, and the lower section comprises from 5% to 20% of the theoretical trays of the column. The upper and lower sections are thermally coupled. Thermally coupled is the term for the energy advantage or thermodynamic advantage for the use of energy in the reboiler and condenser for a single DWC over a traditional cascade of two columns. Separation of the mixture across a cascade of two columns incurs more energy losses than the corresponding separation performed in a single DWC configured for the same or substantially the same feed and product streams. In some embodiments, there is limited thermal exchange across the partition, but this is normally small and effort is typically taken to minimize this heat transfer. The DWC internal partition may or may not be located in the center of the tower and may also not be in the same position relative to the center point of the tower across the entire height of the column where the partition is applied. For example, the product side of the walled section of the tower may be narrower below the feed trays or product tray of the tower. This is a normal part of final engineering design of a DWC as would be understood by one skilled in the art. The main DWC tower diameter may vary (may not be uniform diameter) over the height of the tower. This part of the normal design of any distillation column and depends on normal engineering hydraulic and pneumatic considerations. At the top of the DWC internal wall, either a fixed or full takeoff tray can be used depending on the intent of how the tower is to be operated. Full takeoff trays are normally preferred to allow a wider operating window for a DWC including turn-down (i.e., the ability to effectively operate a distillation tower at reduced feed rates-less efficient but allows production under non-ideal conditions typical of startups, during upsets in other parts of the plant, etc.). Other known devices, techniques, or internal configurations can also be used to control DWC liquid or gas ratios at the wall for operation optimization or flexibility.

A first feed stream 134 and a second feed stream 172 are introduced to the feed side of the intermediate section 1533. The first and second feed streams comprise EB, MBA, and ACP.

A third feed stream 1546, comprising MBA and ACP, is introduced to the lower section 1535 of DWC 1530. Distillation conditions are implemented within DWC 1530 sufficient to permit withdrawal of: an overhead product stream 152a, comprising EB; an intermediate product stream 154a, comprising MBA and ACP, wherein the intermediate product stream is withdrawn from the product side of the intermediate section; and a bottoms product stream 1536a, comprising MBA.

In some embodiments, EB/MBA recovery unit 150a is integrated into a POSM process 100 as shown in FIG. 1. In some embodiments, a process including DWC 1530 further comprises adding the overhead product stream 152a and oxygen 101 (FIG. 1) to an oxidation reaction zone 110 (FIG. 1) to form an oxidation product 112 (FIG. 1) comprising ethylbenzene hydroperoxide.

In some embodiments, a process including DWC 1530 further comprises dehydrating the intermediate product stream 154a in dehydration zone 160 (FIG. 1) to produce a styrene product stream 162 (FIG. 1).

In some embodiments, a process including DWC 1530 further comprises producing the third feed stream 1546 by distilling the bottoms product stream 1536a to form a bottoms product overhead stream 1546 and a bottoms product heavy stream 156 and recycling the bottoms product overhead stream as the third feed stream 1546 to the DWC.

In some embodiments, a process including DWC 1530 further comprises producing the first feed stream 134 by catalytically reacting feed streams 122 (FIG. 1), comprising ethylbenzene hydroperoxide, with propylene 102 (FIG. 1) to form a reaction product comprising propylene oxide. The reaction product is treated to neutralize acidic materials and to remove epoxidation catalyst to form a crude propylene oxide product. The crude propylene oxide product is subjected to one or more distillation steps to produce a light stream 132 (FIG. 1), comprising unreacted propylene and propylene oxide, and a heavy stream 134 (FIG. 1). The heavy stream 134 (FIG. 1) is routed to EB/MBA recovery unit 150a.

In some embodiments, a process including DWC 1530 further comprises producing the second feed stream 172 by treating the intermediate product stream 154a in a dehydration zone 160 (FIG. 1) to form a styrene product stream 162 (FIG. 1) and a dehydration byproducts stream 164 (FIG. 1). The dehydration byproducts stream 164 (FIG. 1) and hydrogen 108 (FIG. 1) are added to a hydrogenation zone 170 (FIG. 1) to form the second feed stream 172.

In some embodiments, the first feed stream 134 comprises EB in the range of from 60 wt %, 55 wt %, or 50 wt % to 65 wt %, 70 wt %, or 75 wt % and MBA in the range of from 30 wt %, 25 wt %, or 20 wt % to 35 wt %, 40 wt %, or 45 wt %. In some embodiments, the second feed stream 172 comprises EB in the range of from 35 wt %, 40 wt %, or 45 wt % to 50 wt %, 55 wt %, or 60 wt % and MBA in the range of from 40 wt %, 35 wt %, or 30 wt % to 45 wt %, 50 wt %, or 55 wt %. In some embodiments, the third feed stream 1546 comprises MBA in the range of from 85 wt %, 80 wt %, or 75 wt % to 90 wt %, 95 wt %, or 99 wt %.

In some embodiments, the overhead product stream 152a comprises EB in an amount greater than or equal to 90 wt %, greater than or equal to 95 wt %, greater than or equal to 98 wt %, or greater than or equal to 99 wt %. In some embodiments, the intermediate product stream 154a comprises MBA in an amount greater than or equal to 70 wt %, greater than or equal to 75 wt %, greater than or equal to 80 wt %, or greater than or equal to 85 wt %, and acetophenone in an amount up to 1 wt %, up to 0.8 wt %, up to 0.6 wt %, or up to 0.4 wt %. In some embodiments, the bottoms product stream 1536a comprises MBA in an amount greater than or equal to 65 wt %, amount greater than or equal to 70 wt %, greater than or equal to 75 wt %, greater than or equal to 80 wt %, or greater than or equal to 85 wt %.

In some embodiments, the process further comprises withdrawing a fourth product stream 1538, comprising oxygenates including but not limited to aldehydes, from the product side of the intermediate section 1533 at an elevation above the intermediate product stream 154a. In some embodiments, the fourth product stream 1538 comprises benzaldehyde in an amount greater than or equal to 60 wt %, greater than or equal to 70 wt %, greater than or equal to 80 wt %, or greater than or equal to 90 wt %. Under some modes of operation of the divided wall column as described, particularly at startup, it may be desirable to remove an additional small flow of product (stream 1538) to preferentially remove benzaldehyde and/or other oxygenates to prevent accumulation of these minor components in the tower. This flow can vary from 0 up to 0.3 wt %, wherein the wt % is based on the total mass of combined feed streams 134, 172, and 1546.

In some embodiments of the process, distillation conditions comprise operating an overhead condenser loop 1532 to maintain a pressure at the upper end of the upper section 1531 in the range of from 0.1 bar-a, 0.2 bar-a, 0.3 bar-a, or 0.4 bar-a to 0.7 bar-a, 0.8 bar-a, 0.9, or 1.0 bar-a.

In some embodiments of the process, distillation conditions comprise operating an overhead condenser loop 1532 to maintain a reflux ratio is in the range of from 0.8:1 to 1.5:1, from 0.9:1 to 1.4:1, from 1.0:1 to 1.3:1, or from 1.1:1 to 1.2:1. In some embodiments of the process, distillation conditions comprise operating a bottoms reboiler loop 1534 to maintain a mass ratio of bottoms product to total feed of from 0.01:1, to 0.30:1, from 0.025:1 to 0.175:1, or from 0.05:1 to 0.15:1.

In some embodiments of the process, the first feed stream 134 enters the DWC 1530 at an elevation above the second feed stream 172, and the amount of MBA in the first feed stream is at least 4 wt %, at least 6 wt %, at least 8 wt %, or at least 10 wt % greater than the amount of methylbenzyl alcohol in the second feed stream.

### System for Co-Production of Propylene Oxide and Styrene Monomer

A system for producing propylene oxide and styrene monomer is disclosed herein. In some embodiments, the system comprises:
a) an oxidation reactor for receiving ethylbenzene and oxygen to produce a crude oxidate;
b) an oxidate concentration unit for receiving the crude oxidate and discharging a concentrated oxidate;
c) an epoxidation reactor for receiving the concentrated oxidate and propylene and discharging a propylene oxide-containing stream;
d) a first distillation unit, comprising one or more distillation columns, for receiving the propylene oxide-containing stream and discharging a light overhead stream comprising propylene oxide and unreacted propylene and a heavy bottoms stream comprising ethylbenzene, methylbenzyl alcohol, acetophenone, and heavy materials; and
e) a dividing-wall distillation column for receiving the heavy bottoms stream and discharging an overhead product stream, an intermediate product stream, and a bottoms product stream, wherein the overhead product stream comprises ethylbenzene, the intermediate product stream comprises methylbenzyl alcohol and acetophenone, and the bottoms product stream comprises methylbenzyl alcohol and heavy materials.

In some embodiments, the system further comprises piping to route the overhead product stream to the oxidation reactor.

In some embodiments, the system further comprises:
a) a dehydration unit for receiving the intermediate product stream and discharging a primary stream comprising styrene monomer and a secondary stream comprising dehydration byproducts;
b) a hydrogenation unit for receiving the secondary stream and hydrogen and discharging a recycle stream, comprising ethylbenzene, methylbenzyl alcohol, and acetophenone; and
c) piping to route the recycle stream to the dividing-wall distillation column.

In some embodiments, the system further comprises:
a) a distillation column for receiving the bottoms product stream to produce a bottoms product overhead stream, comprising methylbenzyl alcohol and acetophenone, and a bottoms product heavy stream; and
b) piping to route the bottoms product overhead stream to dividing-wall column.

### Certain Embodiments

Processes and systems disclosed herein utilize a dividing-wall column (DWC). The DWC has an upper section, an intermediate section, and a lower section, wherein the intermediate section comprises a vertical partition which divides the intermediate section into a feed side and a product side. A first feed stream and a second feed stream are introduced to the feed side of the intermediate section of the DWC, wherein the first and second feed streams comprise ethylbenzene, methylbenzyl alcohol, and acetophenone. A third feed stream is introduced to the lower section of the DWC, wherein the third stream comprises methylbenzyl alcohol and acetophenone. Distillation conditions are implemented within the DWC to facilitate withdrawal of an overhead product stream, an intermediate product stream, and a bottoms product stream. The overhead product stream comprises ethylbenzene. The intermediate product stream comprises methylbenzyl alcohol and acetophenone and is withdrawn from the product side of the intermediate section. The bottoms product stream comprises methylbenzyl alcohol and heavy materials.

In some embodiments of the process, the foregoing process for co-producing propylene oxide and styrene monomer further comprises one or more of the following:
a) adding the overhead product stream and oxygen to an oxidation reaction zone to form an oxidation product comprising ethylbenzene hydroperoxide;
b) dehydrating the intermediate product stream to produce a styrene product stream;
c) producing the third feed stream by:
   i) distilling the bottoms product stream to form a bottoms product overhead stream and a bottoms product heavy stream; and
   ii) recycling the bottoms product overhead stream as the third feed stream to the DWC;
d) producing the first feed stream by:
   i) catalytically reacting feed streams comprising ethylbenzene hydroperoxide with propylene to form a reaction product comprising propylene oxide;
   ii) treating the reaction product to neutralize acidic materials and to remove epoxidation catalyst to form a crude propylene oxide product;
   iii) subjecting the crude propylene oxide product to one or more distillation steps and producing a light stream comprising unreacted propylene and propylene oxide and a heavy stream; and
   iv) withdrawing the heavy stream as the first feed stream; and
e) producing the second feed stream by:
   i) treating the intermediate product stream in a dehydration zone to form a styrene product stream and a dehydration byproducts stream; and
   ii) adding the dehydration byproducts stream and hydrogen to a hydrogenation zone to form the second feed stream.

In some embodiments of the process, a process according to any one of the foregoing embodiments of the process for co-producing propylene oxide and styrene monomer is further characterized by one or more of the following:
a) the first feed stream comprises ethylbenzene in the range of from 60 wt %, 55 wt %, or 50 wt % to 65 wt %, 70 wt %, or 75 wt % and methylbenzyl alcohol in the range of from 30 wt %, 25 wt %, or 20 wt % to 35 wt %, 40 wt %, or 45 wt %;
b) the second feed stream comprises ethylbenzene in the range of from 35 wt %, 40 wt %, or 45 wt % to 50 wt %, 55 wt %, or 60 wt % and methylbenzyl alcohol in the range of from 40 wt %, 35 wt %, or 30 wt % to 45 wt %, 50 wt %, or 55 wt %;
c) the third feed stream comprises methylbenzyl alcohol in the range of from 85 wt %, 80 wt %, or 75 wt % to 90 wt %, 95 wt %, or 99 wt %; or
d) the overhead product stream comprises ethylbenzene in an amount greater than or equal to 90 wt %, greater than or equal to 95 wt %, greater than or equal to 98 wt %, or greater than or equal to 99 wt %;
e) the intermediate product stream comprises methylbenzyl alcohol in an amount greater than or equal to 70 wt %, greater than or equal to 75 wt %, greater than or equal to 80 wt %, or greater than or equal to 85 wt %, and acetophenone in an amount up to 1 wt %, up to 0.8 wt %, up to 0.6 wt %, or up to 0.4 wt %; and
f) the bottoms product stream comprises methylbenzyl alcohol in an amount greater than or equal to 70 wt %, greater than or equal to 75 wt %, greater than or equal to 80 wt %, or greater than or equal to 85 wt %.

In some embodiments of the process, a process according to any one of the foregoing embodiments of the process for co-producing propylene oxide and styrene monomer further comprises withdrawing a fourth product stream from the product side of the intermediate section at an elevation above the intermediate product stream. In further embodiments, the fourth product stream comprises benzaldehyde in an amount greater than or equal to 60 wt %, greater than or equal to 70 wt %, greater than or equal to 80 wt %, or greater than or equal to 90 wt %.

Additional embodiments of the process include any one of the foregoing embodiments of the process for co-producing propylene oxide and styrene monomer wherein distillation conditions comprise one or more of:
a) operating an overhead condenser loop to maintain a pressure at the upper end of the upper section in the range of from 0.1 bar-a, 0.2 bar-a, 0.3 bar-a, or 0.4 bar-a to 0.7 bar-a, 0.8 bar-a, 0.9, or 1.0 bar-a;
b) operating an overhead condenser loop to maintain a reflux ratio is in the range of from 0.8:1 to 1.5:1, from 0.9:1 to 1.4:1, from 1.0:1 to 1.3:1, or from 1.1:1 to 1.2:1; and
c) operating a bottoms reboiler loop to maintain a mass ratio of bottoms product to total feed of from 0.01:1, to 0.30:1, from 0.025:1 to 0.175:1, or from 0.05:1 to 0.15:1.

In some embodiments of the process, a process according to any one of the foregoing embodiments of the process for co-producing propylene oxide and styrene monomer is further characterized by one or more of the following:
a) the DWC comprises trays, packing, or combination thereof sufficient to implement theoretical stages in the range of from 20 to 50, from 22 to 45, from 24 to 40, or from 26 to 35, wherein in further embodiments, the partition extends to span from 10 to 20, from 11 to 18, or from 12 to 16 of these theoretical stages;
b) the upper section comprises from 25% to 50% of the theoretical trays of the DWC, the intermediate section comprises from 30% to 70% of the theoretical trays of the DWC, and the lower section comprises from 20% to 5% of the theoretical trays of the DWC; and
c) the first feed stream enters the DWC at an elevation above the second feed stream, and the amount of MBA in the first feed stream is at least 4 wt %, at least 6 wt %, at least 8 wt %, or at least 10 wt % greater than the amount of MBA in the second feed stream.

In some embodiments, a system for producing propylene oxide and styrene monomer comprises:
a) an oxidation reactor for receiving ethylbenzene and oxygen to produce a crude oxidate;
b) an oxidate concentration unit for receiving the crude oxidate and discharging a concentrated oxidate;
c) an epoxidation reactor for receiving the concentrated oxidate and propylene and discharging a propylene oxide-containing stream;
d) a first distillation unit for receiving the propylene oxide-containing stream and discharging a light overhead stream comprising propylene oxide and unreacted propylene and a heavy bottoms stream comprising ethylbenzene, methylbenzyl alcohol, acetophenone, and heavy materials; and
e) a dividing-wall distillation column for receiving the heavy bottoms stream and discharging an overhead product stream, a first intermediate product stream, optionally a second intermediate stream, and a bottoms product stream, wherein the overhead product stream comprises ethylbenzene, the first intermediate product stream comprises methylbenzyl alcohol and acetophenone, the second intermediate product stream comprises benzaldehyde, and the bottoms product stream comprises methylbenzyl alcohol and heavy materials.

In some embodiments of the system, the foregoing system for co-producing propylene oxide and styrene monomer further comprises piping to route the second overhead stream to the oxidation reactor.

In some embodiments of the system, a system according to any one of the foregoing embodiments of the system for co-producing propylene oxide and styrene monomer further comprises:
a) a dehydration unit for receiving the intermediate product stream and discharging a primary stream comprising styrene monomer and a secondary stream comprising dehydration byproducts;
b) a hydrogenation unit for receiving the secondary stream and hydrogen and discharging a recycle stream, comprising ethylbenzene, methylbenzyl alcohol, and acetophenone; and
c) piping to route the recycle stream to the dividing-wall distillation column.

In some embodiments of the system, a system according to any one of the foregoing embodiments of the system for co-producing propylene oxide and styrene monomer further comprises:
a) a distillation column for receiving the bottoms product stream to produce a bottoms product overhead stream, comprising methylbenzyl alcohol and acetophenone, and a bottoms product heavy stream; and
b) piping to route the bottoms product overhead stream to dividing-wall column.

The presently disclosed process and apparatus are exemplified with respect to the examples below. These examples are included to demonstrate embodiments of the appended claims. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Examples 1 and 2

An Aspen computer simulation (ASPEN Plus V12 steady-state simulation) of process stream compositions and process conditions was used to simulate a comparative Example 1 and inventive Example 2. Model output for comparative Example 1 correlates to streams identified in FIG. 2. Model output for inventive Example 2 correlates to streams identified in FIG. 3. In the simulations, the 2-column configuration had about 32 theoretical stages without including reboilers or condensers. The DWC had about 32 stages (6% less than 2-column configuration) including the reboiler and condenser, or 30 theoretical stages within the main tower shell. The wall section extends across 13 of these stages in the tower. For the DWC example, numbered from the top, not including condenser or reboiler, stages 1-13 were above the partition, stages 14-26 spanned the partition, and stages 27-30 were below the partition.

A dividing-wall column (DWC) was surprisingly and successfully applied to a complex high-purity chemical separation that is outside of usual DWC applications/separations, such as in refining. In this case, three distinct feed streams were fed to a single DWC tower to produce a first product stream rich in ethylbenzene (EB), a second product stream rich in the mixture of acetophenone (ACP) and methylbenzyl alcohol (MBA), and a third product stream comprising MBA and heavy components. In the past, these three product streams were fed to a cascade of two separate distillation towers series to produce the same three product streams.

A first feed stream is sourced from an upstream unit where crude propylene oxide (PO) is distilled to recover PO and other light components and produce a PO crude bottoms stream. The other two feeds are sourced from other recycle streams within the POSM process and are fed to this distillation train to recover additional ACP and MBA. These three feed streams are processed to recover MBA for production of styrene monomer and EB for feed to the oxidation unit. In either the 2-column scenario or the single DWC scenario, a bottoms stream is further distilled to recover MBA for use in the POSM process and produce a heavy hydrocarbon stream to be processed outside the POSM process.

At the same product purity, the DWC product streams meet all intended specifications associated with the traditional 2-column separation train. Additionally, the DWC configuration met quality requirements with less equipment, expected to result in reduced capital and/or maintenance cost than the 2-column configuration. Based on the examples herein, it has been estimated new-build capital costs may be reduced as much as 13% when comparing installation of a DWC system compared to the corresponding traditional two-column system, wherein significant savings are achieved by elimination of additional ancillary equipment required for the two-column configuration. The DWC configuration allowed: the total number of theoretical stages to be reduced by 6%; eliminated the need for a condenser loop, a reboiler loop, and other intermediate equipment along with elimination of the second column; and reduced reboiler duty by 12% without a need to change steam quality (i.e., no need to shift from medium pressure to high pressure steam).

With the same flows and product purity specifications, the DWC configuration is believed to have no impact on upstream facilities, downstream facilities, or internal process recycle streams. However, the DWC configuration may offer flexibility (e.g., size, placement, operation debottleneck, and/or number of partitions) and/or permit adjustment and/or optimization of an overall POSM process towards different targets.

### Example 1

Comparative Example 1 demonstrates an embodiment wherein first feed stream 134 (PO column bottoms) and second feed stream 172 (hydrogenate) are introduced to distillation column 1510. Overhead product stream 152 and bottoms stream 1516 are withdrawn from distillation column 1510. Bottoms stream 1516 and third feed stream 1546 (MBA stripper distillate) are introduced to distillation column 1520. Overhead product stream 154 and bottoms stream 1536 are withdrawn from distillation column 1520.

### Example 2

Inventive Example 2 demonstrates an embodiment wherein first feed stream 134 (PO column bottoms), second feed stream 172 (hydrogenate), and third feed stream 1546 (MBA stripper distillate) are introduced to DWC 1530. Overhead product stream 152a, intermediate product stream 154a, and bottoms product stream 1536a are withdrawn from DWC 1530.

### Results

Table 1 shows the composition of feed streams 134, 172, and 1546 used in simulations for Examples 1 and 2.

TABLE 1

| Component | Feed Stream | | |
|---|---|---|---|
| (mass fraction) | 134 | 172 | 1546 |
| N₂ | 0.00E+00 | 0.00E+00 | 0.00E+00 |
| O₂ | 0.00E+00 | 0.00E+00 | 0.00E+00 |
| methane | 0.00E+00 | 1.20E−04 | 0.00E+00 |
| water | 0.00E+00 | 1.60E−03 | 0.00E+00 |
| benzene and toluene | 4.17E−04 | 1.78E−03 | 0.00E+00 |
| EB | 6.27E−01 | 4.99E−01 | 0.00E+00 |
| xylene | 6.72E−04 | 7.53E−04 | 0.00E+00 |
| styrene | 7.44E−04 | 0.00E+00 | 0.00E+00 |
| cumene | 5.77E−04 | 6.93E−04 | 0.00E+00 |
| benzaldehyde | 1.18E−03 | 9.03E−05 | 0.00E+00 |
| phenol | 4.92E−04 | 1.08E−03 | 0.00E+00 |
| propylene glycol | 2.68E−03 | 0.00E+00 | 0.00E+00 |
| ACP | 3.68E−02 | 3.37E−02 | 7.93E−03 |
| MBA | 3.14E−01 | 4.15E−01 | 8.79E−01 |
| Other* | 1.50E−02 | 4.61E−02 | 1.13E−01 |
| NaOH | 0.00E+00 | 0.00E+00 | 0.00E+00 |
| Na salts | 1.90E−04 | 0.00E+00 | 0.00E+00 |

*Other heavy components including oxygenates such as alcohols, aldehydes, carbinols, and acids

Table 2 shows the mass fraction of all constituents of the compositions of product streams 152, 154, and 1536 (FIG. 2) produced by the simulation model for Example 1 and product streams 152a, 154a, and 1536a produced by the simulation model for Example 2 (FIG. 3).

TABLE 2

| Component | 2-Column Product Stream | | | DWC Product Stream | | |
|---|---|---|---|---|---|---|
| (mass fraction) | 152 | 154 | 1536 | 152a | 154a | 1536a |
| N₂ | 0.00E+00 | 0.00E+00 | 0.00E+00 | 0.00E+00 | 0.00E+00 | 0.00E+00 |
| O₂ | 0.00E+00 | 0.00E+00 | 0.00E+00 | 0.00E+00 | 0.00E+00 | 0.00E+00 |
| methane | 0.00E+00 | 0.00E+00 | 0.00E+00 | 0.00E+00 | 0.00E+00 | 0.00E+00 |
| water | 1.91E−04 | 0.00E+00 | 0.00E+00 | 2.55E−04 | 2.62E−14 | 2.44E−19 |
| benzene and toluene | 8.93E−04 | 0.00E+00 | 0.00E+00 | 8.88E−04 | 1.09E−10 | 1.59E−14 |
| EB | 9.92E−01 | 3.71E−05 | 0.00E+00 | 9.92E−01 | 1.51E−04 | 3.23E−09 |
| xylene | 1.09E−03 | 9.27E−06 | 0.00E+00 | 1.09E−03 | 7.83E−06 | 1.65E−09 |
| styrene | 1.08E−03 | 0.00E+00 | 0.00E+00 | 1.07E−03 | 1.92E−05 | 3.12E−10 |
| cumene | 8.59E−04 | 1.39E−04 | 0.00E+00 | 4.15E−04 | 8.99E−04 | 1.75E−07 |
| benzaldehyde | 1.38E−04 | 2.58E−03 | 0.00E+00 | 6.92E−06 | 2.90E−03 | 2.48E−05 |
| phenol | 0.00E+00 | 1.49E−03 | 0.00E+00 | 2.42E−12 | 1.48E−03 | 7.69E−05 |
| propylene glycol | 3.14E−03 | 8.72E−04 | 0.00E+00 | 3.86E−03 | 6.28E−05 | 1.17E−06 |
| ACP | 3.34E−05 | 9.89E−02 | 6.45E−03 | 2.15E−09 | 9.65E−02 | 2.22E−02 |
| MBA | 4.30E−04 | 8.78E−01 | 7.14E−01 | 2.81E−08 | 8.76E−01 | 7.13E−01 |
| Other* | 4.77E−06 | 1.83E−02 | 2.77E−01 | 6.60E−17 | 2.15E−02 | 2.63E−01 |
| Na salts | 0.00E+00 | 0.00E+00 | 2.83E−03 | 4.82E−56 | 3.20E−21 | 2.79E−03 |

*Other heavy components including oxygenates such as alcohols, aldehydes, carbinols, and acids

Table 3 provides a synopsis of Table 2 to focus on a direct comparison of the three target product streams. Scientific notation of the mass fractions was converted to weight percentages, and all components that were present at less than 1 wt % were removed. Columns 1 and 2 show that the EB content of the overhead product streams for Examples 1 and 2 are substantially equivalent. Columns 3 and 4 show that the MBA content and ACP content of the intermediate product streams for Examples 1 and 2 are substantially equivalent. Columns 5 and 6 show that the MBA content and other (heavies) content of the bottoms product streams for Examples 1 and 2 are substantially equivalent.

TABLE 3

| | Overhead Product | | Intermediate Product | | Bottoms Product | |
|---|---|---|---|---|---|---|
| | (EB) | | (MBA/ACP) | | (MBA/Heavy) | |
| Component | Stream | | | | | |
| (wt %) | 152 | 152a | 154 | 154a | 1526 | 1526a |
| EB | 99.21% | 99.24% | 0.00% | 0.02% | 0.00% | 0.00% |
| ACP | 0.00% | 0.00% | 9.89% | 9.65% | 0.65% | 2.22% |
| MBA | 0.04% | 0.00% | 87.77% | 87.65% | 71.39% | 71.27% |
| benzyl alcohol | 0.00% | 0.00% | 1.10% | 1.22% | 2.18% | 1.44% |
| PEA | 0.00% | 0.00% | 0.32% | 0.56% | 4.90% | 3.38% |
| others | 0.00% | 0.00% | 0.03% | 0.00% | 19.70% | 19.66% |

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, means, methods, and/or steps described in the specification. As one of the ordinary skill in the art will readily appreciate from the disclosure of the present invention, processes, machines, means, methods, and/or steps, presently existing or later to be developed that perform substantially the same function or achieve substantially the same result as the corresponding embodiments described herein, may be utilized according to the present invention. Accordingly, the appended claims are intended to include within their scope such processes, machines, means, methods, and/or steps.

What is claimed is:

1. A process comprising:
a) providing a dividing-wall column (DWC) having an upper section, an intermediate section, and a lower section, wherein the intermediate section comprises a vertical partition which divides the intermediate section into a feed side and a product side;
b) introducing
i) a first feed stream and a second feed stream to the feed side of the intermediate section, wherein the first and second feed streams comprise ethylbenzene, methylbenzyl alcohol, and acetophenone; and
ii) a third feed stream to the lower section, wherein the third stream comprises methylbenzyl alcohol and acetophenone;
c) implementing distillation conditions within the DWC;
d) withdrawing
i) an overhead product stream, comprising ethylbenzene;
ii) an intermediate product stream, comprising methylbenzyl alcohol and acetophenone, wherein the intermediate product stream is withdrawn from the product side of the intermediate section; and
iii) a bottoms product stream, comprising methylbenzyl alcohol.

2. The process of claim 1, further comprising adding the overhead product stream and oxygen to an oxidation reaction zone to form an oxidation product comprising ethylbenzene hydroperoxide.

3. The process of claim 1, further comprising dehydrating the intermediate product stream to produce a styrene product stream.

4. The process of claim 1, further comprising producing the third feed stream by:
a) distilling the bottoms product stream to form a bottoms product overhead stream and a bottoms product heavy stream; and
b) recycling the bottoms product overhead stream as the third feed stream to the DWC.

5. The process of claim 1, further comprising producing the first feed stream by:
a) catalytically reacting feed streams comprising ethylbenzene hydroperoxide with propylene to form a reaction product comprising propylene oxide;
b) treating the reaction product to neutralize acidic materials and to remove epoxidation catalyst to form a crude propylene oxide product;
c) subjecting the crude propylene oxide product to one or more distillation steps and producing a light stream comprising unreacted propylene and propylene oxide and a heavy stream; and
d) withdrawing the heavy stream as the first feed stream.

6. The process of claim 1, further comprising producing the second feed stream by:
a) treating the intermediate product stream in a dehydration zone to form a styrene product stream and a dehydration byproducts stream; and
b) adding the dehydration byproducts stream and hydrogen to a hydrogenation zone to form the second feed stream.

7. The process of claim 1, wherein:
a) the first feed stream comprises ethylbenzene in the range of from 50 wt % to 75 wt % and methylbenzyl alcohol in the range of from 20 wt % to 45 wt %;
b) the second feed stream comprises ethylbenzene in the range of from 35 wt % to 60 wt % and methylbenzyl alcohol in the range of from 30 wt % to 55 wt %;
c) the third feed stream comprises methylbenzyl alcohol in the range of from 75 wt % to 99 wt %; or
d) a combination thereof.

8. The process of claim 1, wherein:
a) the overhead product stream comprises ethylbenzene in an amount greater than or equal to 90 wt %, greater than or equal to 95 wt %, greater than or equal to 98 wt %, or greater than or equal to 99 wt %;
b) the intermediate product stream comprises methylbenzyl alcohol in an amount greater than or equal to 70 wt %, and acetophenone in an amount up to 1 wt %;
c) the bottoms product stream comprises methylbenzyl alcohol in an amount greater than or equal to 70 wt %; or
d) a combination thereof.

9. The process of claim 1, further comprising withdrawing a fourth product stream from the product side of the intermediate section at an elevation above the intermediate product stream.

10. The process of claim 9, wherein the fourth product stream comprises benzaldehyde in an amount greater than or equal to 60 wt %.

11. The process of claim 1, wherein distillation conditions comprise operating an overhead condenser loop to maintain a pressure at the upper end of the upper section in the range of from 0.1 bar-a to 1.0 bar-a.

12. The process of claim 1, wherein distillation conditions comprise:
a) operating an overhead condenser loop to maintain a reflux ratio is in the range of from 0.8:1 to 1.5:1;
b) operating a bottoms reboiler loop to maintain a mass ratio of bottoms product to total feed of from 0.01:1 to 0.30:1; or
c) a combination thereof.

13. The process of claim 1, wherein the DWC comprises trays, packing, or combination thereof sufficient to implement theoretical stages in the range of from 20 to 50.

14. The process of claim 13, wherein the partition extends to span across 10 to 20 of these theoretical stages.

15. The process of claim 1, wherein the upper section comprises from 25% to 50% of the theoretical trays of the DWC, the intermediate section comprises from 30% to 70% of the theoretical trays of the DWC, and the lower section comprises from 20% to 5% of the theoretical trays of the DWC.

16. The process of claim 1, wherein:
a) the first feed stream enters the DWC at an elevation above the second feed stream; and
b) the amount of methylbenzyl alcohol in the first feed stream is at least 4 wt %, greater than the amount of methylbenzyl alcohol in the second feed stream.

17. A system for producing propylene oxide and styrene monomer, the system comprising:
a) an oxidation reactor for receiving ethylbenzene and oxygen to produce a crude oxidate;
b) an oxidate concentration unit for receiving the crude oxidate and discharging a concentrated oxidate;
c) an epoxidation reactor for receiving the concentrated oxidate and propylene and discharging a propylene oxide-containing stream;
d) a first distillation unit for receiving the propylene oxide-containing stream and discharging a light overhead stream comprising propylene oxide and unreacted propylene and a heavy bottoms stream comprising ethylbenzene, methylbenzyl alcohol, acetophenone, and heavy materials; and
e) a dividing-wall distillation column for receiving the heavy bottoms stream and discharging an overhead product stream, a first intermediate product stream, optionally a second intermediate stream, and a bottoms product stream, wherein the overhead product stream comprises ethylbenzene, the first intermediate product stream comprises methylbenzyl alcohol and acetophenone, the second intermediate product stream comprises benzaldehyde, and the bottoms product stream comprises methylbenzyl alcohol and heavy materials.

18. The system of claim 17, further comprising piping to route the second overhead stream to the oxidation reactor.

19. The system of claim 17, further comprising:
a) a dehydration unit for receiving the intermediate product stream and discharging a primary stream comprising styrene monomer and a secondary stream comprising dehydration byproducts;
b) a hydrogenation unit for receiving the secondary stream and hydrogen and discharging a recycle stream, comprising ethylbenzene, methylbenzyl alcohol, and acetophenone; and
c) piping to route the recycle stream to the dividing-wall distillation column.

20. The system of claim 17, further comprising:
a) a distillation column for receiving the bottoms product stream to produce a bottoms product overhead stream, comprising methylbenzyl alcohol and acetophenone, and a bottoms product heavy stream; and
b) piping to route the bottoms product overhead stream to dividing-wall column.
